(19) United States
(12) Patent Application Publication
(10) Pub. No.: US 2005/0033316 A1
(43) Pub. Date: Feb. 10, 2005

(54) Ultrasonic skin cleaner

(76) Inventor: M. Glen Kertz, Orange, TX (US)

Correspondence Address: CONLEY ROSE, P.C., P. O. BOX 3267, HOUSTON, TX, 77253-3267, US

(21) Appl. No.: 10/890,041

(22) Filed: Jul. 13, 2004

Related U.S. Application Data
(60) Provisional application No. 60/487,560, filed on Jul. 14, 2003.

(51) Int. Cl. A61B 17/50

(52) U.S. Cl. 606/131

(57) ABSTRACT

Methods and apparatus for treating the skin using an apparatus comprising a first vibration generator disposed within a body and coupled to a transfer rod. The apparatus may comprise a second vibration generator to provide ultrasonic vibrations in multiple planes. The transfer rod has a first end extended from said body. A head is attached to the first end of said transfer rod. A pad is removably connected to said head and provides a skin contacting surface that can be easily replaced or removed for cleaning.

### CROSS-REFERENCE TO RELATED APPLICATIONS

The present application claims priority to U.S. Provisional Application No. 60/487,560, filed Jul. 14, 2003, titled “An Ultrasonic Skin Cleaner,” and hereby incorporated herein by reference for all purposes.

### STATEMENT REGARDING FEDERALLY SPONSORED RESEARCH OR DEVELOPMENT

Not Applicable.

### BACKGROUND

The present invention relates generally to methods and apparatus for cleaning human skin. More particularly, the present invention relates to methods and apparatus that use ultrasonic vibrations to clean human skin.

Ultrasonic vibrations and ultrasound have been used on human skin to perform a variety of tasks, including: softening the thrombus, removing scars after acne is treated, removing abnormal skin color, decomposing abnormal pigment and remove flecks under skin, preventing and removing facial wrinkles and blood stasis, removing “eye-sack” and “black eye-rim,” softening nodules to treat acne, and treating sclerosis and parchment. Ultrasonic vibrations have also been claimed to improve skin tone as well as underlying muscle tone.

In most cases, the application of ultrasonic vibrations to human skin has been done in a spa or clinic setting. These treatments commonly use an ultrasonic transducer that produces high-frequency sound waves up to and including 1 MHz. The ultrasonic transducer transfers vibrations through a metal plate that is applied directly to the skin. The transducer is attached via a cord to a stationary base unit that powers the transducer. A typical treatment may last from 15 to 30 minutes and can cost several hundred to several thousand dollars.

There are also a number of hand-held, home-use devices that employ ultrasound for treating the skin. These hand-held devices include of one or more metal plates that are applied directly to the area of skin that is to be treated. An ultrasonic transducer activates the metal plates and the ultrasonic vibrations pass into the skin and surrounding tissue. These hand-held units are designed to help reduce the visible effects of aging and are sold as a low cost alternative to surgical face-lifts. The units do have great beneficial effects on the overall health of skin, in particular the skin of the face and neck. They can reduce visible wrinkles, smooth scars and increase general muscle tone of under-laying subcutaneous tissue. Unfortunately, some of these units also pose a slight health risk in that the energy that they produce may be damaging to the retina and optic nerve of the human eye, should the user make accidental contact with the eye. This health risk has limited the availability of these products in many countries.

Most of the currently available ultrasonic skin care products are designed to rejuvenate, or improve the overall quality of, the skin but are not specifically designed to act as a daily cleaning appliance. In some treatments ultrasonic energy is used to drive beneficial compounds into the surface of the skin. This is known as “sonophoresis.” However, unwanted impurities found on the surface of the skin may also be driven in by the use of ultrasound. For this reason, it is critical that the surface of the skin to be treated to be free of any foreign material, including, but not limited to, dust, grime, dead skin, excess body oil or other contaminates that may be found on the surface of exposed skin. Therefore, it is often recommended to pre-clean the skin before application of the ultrasonic device as the high frequency sound may in fact drive surface impurities deeper into the skin surface.

Accordingly, it would be desirable to have an ultrasonic skin cleaning system that is intended for daily personal use. Thus, there remains a need to develop methods and apparatus for ultrasonic skin cleaning, which overcome some of the foregoing difficulties while providing more advantageous overall results.

### SUMMARY OF THE PREFERRED EMBODIMENTS

The embodiments of the present invention are directed toward methods and apparatus for treating the skin using an apparatus comprising a first vibration generator disposed within a body and coupled to a transfer rod. The apparatus may comprise a second vibration generator to provide ultrasonic vibrations in multiple planes. The transfer rod has a first end extended from the body. A head is attached to the first end of the transfer rod. A pad is removably connected to the head and provides a skin contacting surface that can be easily replaced or removed for cleaning.

In one embodiment an apparatus for treating skin comprises a transfer rod having a first end extended from a body. A first vibration generator is disposed within the body and is coupled to the transfer rod. A head is attached to the first end of the transfer rod and may be disposed at an angle to the body. A pad is removably connected to the head, such as by a snap ring. A second vibration generator may disposed within the body and also be coupled to the transfer rod. The first vibration generator may operable to vibrate the pad in a first plane and the second vibration generator may be operable to vibrate the pad in a second plane. A battery and a charger may be disposed within the body and electrically coupled to the vibration generators.

In another embodiment, a device comprises a body having a transfer rod extending therefrom and a cleaning base attached to the transfer rod. A first vibration generator is disposed within the body and operable to vibrate the transfer rod in a first plane. A second vibration generator disposed within the body and operable to vibrate the transfer rod in a second plane. A pad may be removably connected to the cleaning base, such as by a snap ring.

Other embodiments include a method of treating skin by attaching a pad to a head mounted to a transfer rod extending from a body, activating an ultrasonic vibration generator disposed within the body so as to vibrate the pad, and applying the vibrating pad to the skin. The ultrasonic vibration generator can be deactivated and the pad removed pad from the head for cleaning or replacing with a new pad. The method may also comprise disposing the body in a recharging base that is connected the recharging base to a power supply. Electrical energy can then be transferred from the power supply to a battery disposed within the body and coupled to the ultrasonic vibration generator.

Other methods for treating skin include applying a head to the skin, wherein the head is mounted to a transfer rod extending from a body and vibrating the head in at least two planes with one or more ultrasonic vibration generators that are disposed within the body and coupled to the transfer rod. A pad may be attached to the head before applying the head to the skin and removed from the head so as to provide a new pad before reapplying to the skin.

Thus, the present invention comprises a combination of features and advantages that enable it to overcome various problems of prior devices. The various characteristics described above, as well as other features, will be readily apparent to those skilled in the art upon reading the following detailed description of the preferred embodiments of the invention, and by referring to the accompanying drawings.

### BRIEF DESCRIPTION OF THE DRAWINGS

For a more detailed description of the preferred embodiment of the present invention, reference will now be made to the accompanying drawings, wherein:

FIG. 1 is a perspective view of an ultrasonic skin cleaner constructed in accordance with one embodiment of the invention;

FIG. 2 is another perspective view of the ultrasonic skin cleaner of FIG. 1;

FIG. 3 is a cross-sectional view of the ultrasonic skin cleaner of FIG. 1;

FIG. 4 is a top view of an ultrasonic skin cleaner recharging base constructed in accordance with embodiments of the invention;

FIG. 5 is a cross-sectional view of the ultrasonic skin cleaner recharging base of FIG. 4; and

FIG. 6 is a perspective view of an ultrasonic skin cleaner cleaning pad constructed in accordance with embodiments of the invention.

### DETAILED DESCRIPTION OF THE PREFERRED EMBODIMENTS

In the description that follows, like parts are marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The present invention is susceptible to embodiments of different forms. There are shown in the drawings, and herein will be described in detail, specific embodiments of the present invention with the understanding that the present disclosure is to be considered an exemplification of the principles of the invention, and is not intended to limit the invention to that illustrated and described herein. It is to be fully recognized that the different teachings of the embodiments discussed below may be employed separately or in any suitable combination to produce desired results.

In particular, various embodiments described herein thus comprise a combination of features and advantages that overcome some of the deficiencies or shortcomings of prior art ultrasonic skin cleaning systems. The various characteristics mentioned above, as well as other features and characteristics described in more detail below, will be readily apparent to those skilled in the art upon reading the following detailed description of preferred embodiments, and by referring to the accompanying drawings.

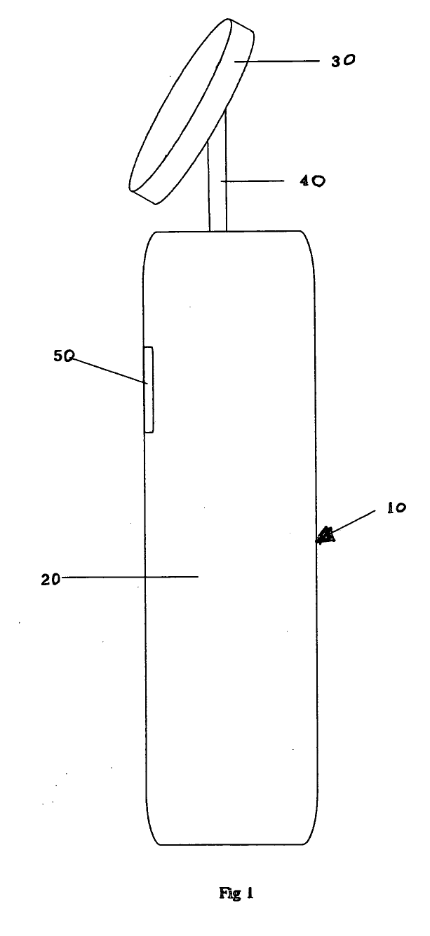

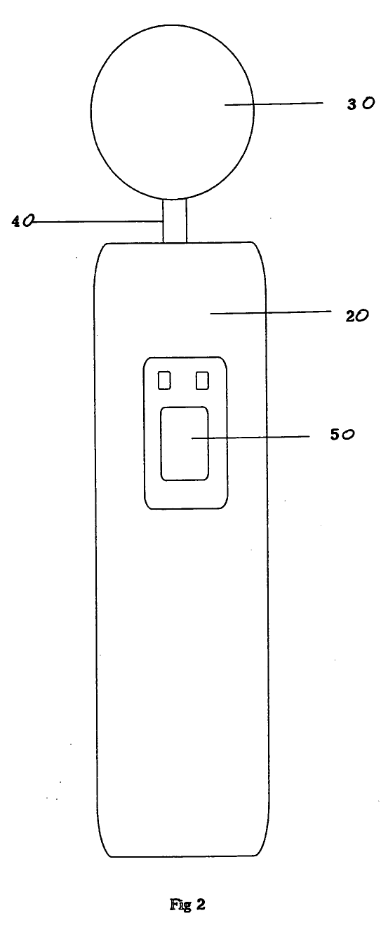

Referring now to FIGS. 1 and 2, ultrasonic skin cleaner 10 comprises main body 20, cleaning head 30, and ultrasonic transfer rod 40. Control panel 50 is mounted on main body 20 and includes a power switch and controls for adjusting the operation of cleaner 10. Main body 20 is sized so as to be hand-held. Cleaning head 30 is at an angle to body 20 in order to allow easy application of the cleaning head to the skin surface. Cleaning head 30 is preferably round in shape, allowing easy contact with contoured skin. Additional shapes for cleaning head 30 may also be used, depending on the desired application.

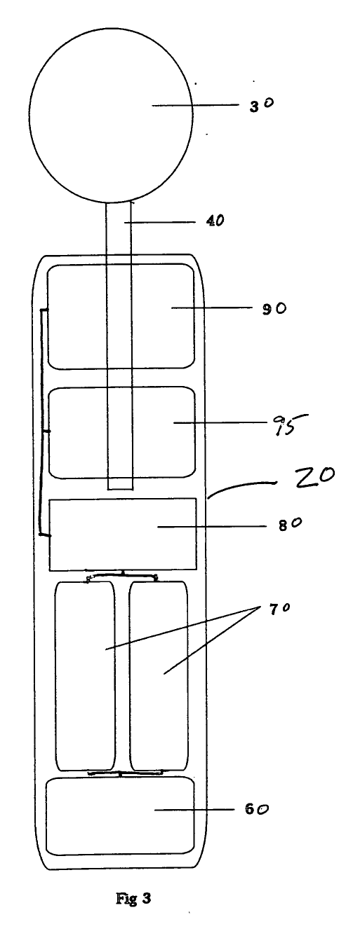

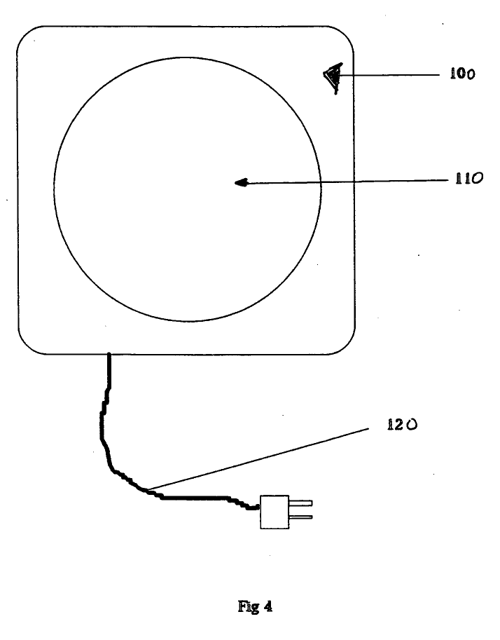

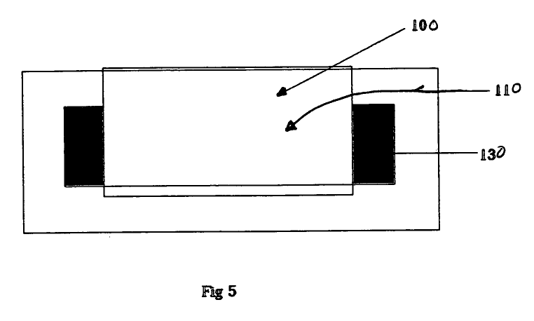

Referring now to FIG. 3, main body 20 houses charger 60, rechargeable batteries 70, electronic control center 80, and ultrasonic vibration generators 90 and 95. Cleaning head 30 is mounted to the protruding end of ultrasonic transfer rod 40. Ultrasonic transfer rod 40 extends into body 20 and is connected to first ultrasonic vibration generator 90 and second ultrasonic vibration generator 95. The frequency and timing of ultrasonic vibration generators 90 and 95 are controlled by electronic control center 80, which receives user input from control panel 50 (see FIGS. 1 and 2). Rechargeable batteries 70 provide power to ultrasonic vibration generators 90 and 95 through electronic control center 80. Charger 60 is coupled to rechargeable batteries 70 and, when connected to a recharging base 100 (see FIGS. 4 and 5), recharges the batteries.

Referring now to FIGS. 4 and 5, recharging base 100 comprises recharging chamber 110, power cord 120, and induction charging coil 130. Recharging chamber 110 provides a recessed area in base 100 that is sized to accept main body 20 (see FIGS. 1-3). Induction charging coil 130 surrounds recharging chamber 110 and provides power to charger 60 when power cord 120 is connected to a power supply (not shown).

Many different skin types require different levels of care in daily cleaning. Providing the optimum daily cleaning regime requires great flexibility in both the type of cleaning agent as well as the physical properties of the cleaning surface. It may be desirous to have a cleaning system that allows the application of other skin care agents, such as moisturizers. These other skin care agents may require a surface pad that is different than that which would be used for cleaning the skin. Further, it is preferred that any cleaning surface be easily cleaned or changed upon each use. Without cleaning or changing, the cleaning surface may harbor a build up of oils and dead skin, thus providing a source of bacterial contamination.

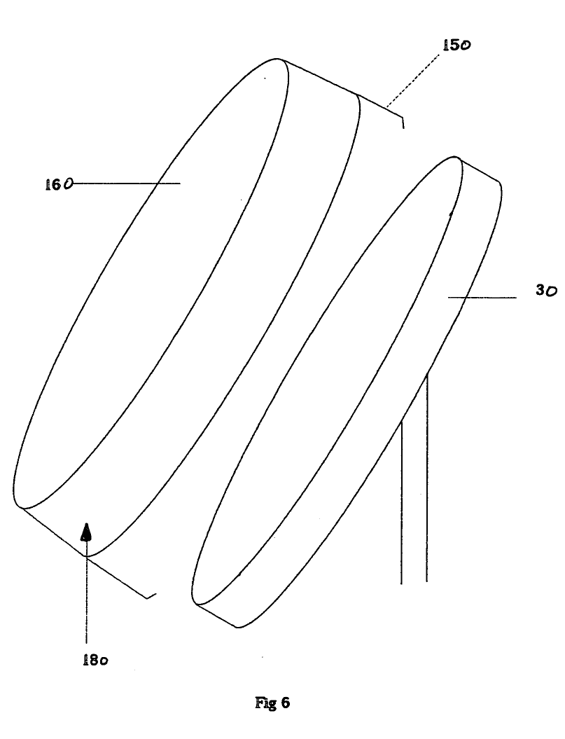

Referring now to FIG. 6, removable cleaning pad 180 comprises attachment ring 150 and cleaning surface 160. Removable cleaning pad 180 attaches to cleaning head 30 via attachment ring 150. Attachment ring 150 may be a compression snap ring easily connected to and disconnected from head 30. Additional attachment methods including an elastic slip cover, grove attachment, and any other form of attachments that would facilitate the easy attachment or removal of cleaning pad 180 to cleaning head 30. Cleaning surface 160 may be made of any natural or artificial fibers or other material as may be suitable for the desired cleaning effect on the skin. The degree of abrasion to the treated skin surface and the depth of the cleaning may be controlled by the nature and composition of the cleaning pad 180.

In operation, a cleaning pad 180 is attached to the cleaning head 30, by attachment ring 150. Cleaning pad 180 may be dampened with water or any desires cleaning agent. Ultrasonic skin cleaner 10 is powered-on by a switch on control panel 50 that activates the electronic control center 80. Control center 80, using power supplied by batteries 70, activates the ultrasonic vibration generators 90 and 95. Ultrasonic vibrations that are generated in the ultrasonic vibration generators 90 and 95 are transferred to the cleaning head 30 and pad 180 via ultrasonic transfer rod 40. Once the unit is powered-on, the surface 160 of cleaning pad 180 is applied to the area of skin that is to be cleaned.

Ultrasonic vibration generators 90 and 95 may be arranged such that their operation causes cleaning head 30 and pad 180 to vibrate in multiple directions. The primary cleaning action of ultrasonic skin cleaner 10 is achieved at the contact between surface 160 of cleaning pad 180 and the surface of the skin. As cleaning pad 180 is applied to the skin, ultrasonic vibrations transferred from cleaning head 30 move the surface 160 of cleaning pad 180 at very high speeds. As cleaning pad 180 is moved back and forth across the area of skin to be cleaned, the high-speed vibrations act to lift deep-seated dirt and grime, dead skin cells, and other foreign matter from the skin. In addition, the high-speed vibrations may abrade the skin and impart a cleaner fresher look to the treated skin.

All vibrations, including ultrasonic vibrations, cause a displacement in any surface that they react with. This displacement can occur in one or more of three dimensions based on how the vibrations are propagated or generated. In basic terms, the vibrations can cause a surface to move, or be displaced, in a classic x, y, or z orientation in three-dimensional space. For the purpose of cleaning the skin, it is advantageous to have a cleaning surface that is moving or displacing in more than one plane. Specifically, it is advantageous to have the cleaning surface moving in both the x and y axis at the same time. By properly timing the generation of specific vibrations, an orbital pattern of movement can be achieved in the cleaning surface. This orbital motion allows greater movement of the surface of the skin, thus resulting in a greater cleaning action.

Ultrasonic vibration generators 90 and 95 may be used to generate ultrasonic vibrations in two planes and thus cause ultrasonic transfer rod 40 to move the cleaning head 30 in multiple dimensions during use. Adjusting the timing of the vibrations in the multiple planes during operation causes a displacement of cleaning head 30 and cleaning pad 180, which is in contact with the skin. Ultrasonic vibration generators 90 and 95 may be ultrasonic transducers, unbalanced electric motors, electromagnets, or any other device capable of generating high-frequency vibrations. In certain embodiments, a single ultrasonic vibration generator may be used to produce unidirectional motion.

Control center 80 may provide for a timed or non-timed treatment period as may be desired by the user. Thus, when electronic control center 80 switches cleaner 10 off, the cleaning pad 180 may be removed for cleaning or replacement. After use, cleaner 10 may be placed into charging base 100 such that rechargeable batteries 70 can be recharged by charger 60 and induction charger coil 130. As an alternative to rechargeable batteries 70, certain embodiments of cleaner 10 may be equipped with replaceable batteries or be connectable to an external power supply.

As described herein, ultrasonic skin cleaner 10 provides a multi-directional ultrasonic skin cleaning appliance comprising a hand held body 20 having a rechargeable battery 70 and a removable and/or replaceable cleaning pad 180. Body 20 and recharging base 100 may also preferably be freestanding and waterproof so as to enable easy and safe storage in the home environment. Thus, ultrasonic skin cleaner 10 allows a user to deep clean the surface of the skin in the privacy of their home on a daily basis. Advantages to this type of deep cleaning would assist in the control of acne, the removal of blemishes, reduction of scar tissue and improvement to the general overall health of the skin that is treated.

In other embodiments, ultrasonic cleaner 10 may have a removable and interchangeable cleaning head. A removable cleaning head could be removed from the transfer rod and replaced with an alternate cleaning head having another shape. The alternate cleaning head would also carry the vibrations from the transfer rod. Interchangeable cleaning heads could also support multiple uses of a single drive unit. For example, one alternate head may be a shaving head including a blade holder. Another alternate head may comprise a shaped, abrasive pad for use in nail care for shaping and/or buffing the nail. Alternate heads may also include heating elements, high-intensity magnets, or other features to improve cleaning performance.

In another embodiment, an ultrasonic cleaner may have a transfer rod that is turned at a 90 from the drive units and have a cleaning head that floats on a gasket. The cleaning head could support removable cleaning pads. This configuration would produce a device that is designed for cleaning large areas of skin. The device would preferably water-proof and could be designed to look similar to a bar of soap.

While preferred embodiments of this invention have been shown and described, modifications thereof can be made by one skilled in the art without departing from the scope or teaching of this invention. The embodiments described herein are exemplary only and are not limiting. Many variations and modifications of the system and apparatus are possible and are within the scope of the invention. For example, the relative dimensions of various parts, the materials from which the various parts are made, and other parameters can be varied, so long as the systems retain the advantages discussed herein. Accordingly, the scope of protection is not limited to the embodiments described herein, but is only limited by the claims that follow, the scope of which shall include all equivalents of the subject matter of the claims.

1. An apparatus for treating skin comprising:
a body;
a transfer rod having a first end extended from said body;
a first vibration generator disposed within said body and coupled to said transfer rod;
a head attached to the first end of said transfer rod; and
a pad removably connected to said head.

2. The apparatus of claim 1 further comprising a second vibration generator disposed within said body and coupled to said transfer rod.

3. The apparatus of claim 2 wherein said first vibration generator is operable to vibrate said pad in a first plane and said second vibration generator is operable to vibrate said pad in a second plane.

4. The apparatus of claim 1 wherein said head is disposed at an angle to said body.

5. The apparatus of claim 1 further comprising a snap ring removably connecting said pad to said head.

6. The apparatus of claim 1 further comprising a battery disposed within said body and electrically coupled to said first vibration generator.

7. The apparatus of claim 6 further comprising a charger disposed within said body and electrically coupled to said battery.

8. The apparatus of claim 7 wherein said charger is operable to receive electric power from an induction coil.

9. The apparatus of claim 1 wherein said body is hand-held.

10. A device comprising;
a body;
a transfer rod extending from said body;
a cleaning head attached to said transfer rod;
a first vibration generator disposed within said body and operable to vibrate said transfer rod in a first plane; and
a second vibration generator disposed within said body and operable to vibrate said transfer rod in a second plane.

11. The device of claim 10 further comprising a pad removably connected to said cleaning head.

12. The device of claim 11 further comprising a snap ring removably connecting said pad to said head.

13. The device of claim 10 wherein said cleaning head is disposed at an angle to said body.

14. The device of claim 10 further comprising a battery disposed within said body and electrically coupled to said vibration generators.

15. The device of claim 14 further comprising a charger disposed within said body and electrically coupled to said battery.

16. The device of claim 15 wherein said charger is operable to receive electric power from an induction coil.

17. The device of claim 10 wherein said body is hand-held.

18. A method of treating skin comprising:
attaching a pad to a head mounted to a transfer rod extending from a body;
activating an ultrasonic vibration generator disposed within the body so as to vibrate the pad;
applying the vibrating pad to the skin;
deactivating the ultrasonic vibration generator; and
removing the pad from the head.

19. The method of claim 18 further comprising replacing the pad with a new pad.

20. The method of claim 18 further comprising cleaning the pad before reattaching the pad to the head.

21. The method of claim 18 further comprising:
disposing the body in a recharging base;
connecting the recharging base to a power supply;
transferring electrical energy from the power supply to a battery disposed within the body and coupled to the ultrasonic vibration generator.

22. The method of claim 18 wherein the pad is vibrated in at least two planes.

23. The method of claim 18 wherein the body is hand-held.

24. A method of treating skin comprising:
applying a head to the skin, wherein the head is mounted to a transfer rod extending from a body; and
vibrating the head in at least two planes with one or more ultrasonic vibration generators that are disposed within the body and coupled to the transfer rod.

25. The method of claim 24 further comprising attaching a pad to the head before applying the head to the skin.

26. The method of claim 25 further comprising removing the pad from the head so as to provide a new pad before reapplying to the skin.

27. The method of claim 24 further comprising:
disposing the body in a recharging base;
connecting the recharging base to a power supply;
transferring electrical energy from the power supply to a battery disposed within the body and coupled to the ultrasonic vibration generator.
